(19) United States
(12) Patent Application Publication
(10) Pub. No.: US 2024/0044369 A1
(43) Pub. Date: Feb. 8, 2024

(54) PROJECTION-BEARING BODY MANUFACTURING METHOD AND PROJECTION-BEARING BODY MANUFACTURING DEVICE

(71) Applicant: LINTEC CORPORATION, Tokyo (JP)

(72) Inventor: Yosuke KOMA, Tokyo (JP)

(73) Assignee: LINTEC CORPORATION, Tokyo (JP)

(21) Appl. No.: 18/244,965

(22) Filed: Sep. 12, 2023

Related U.S. Application Data
(63) Continuation of application No. PCT/JP2021/043183, filed on Nov. 25, 2021.

(30) Foreign Application Priority Data
Mar. 30, 2021 (JP) 2021-058660

(51) Int. Cl. F16C 33/20; B29C 43/02; B29C 71/00

(52) U.S. Cl. CPC F16C 33/208; B29C 43/021; B29C 71/0009; F16C 2208/20; F16C 2220/40; B29C 2043/026

(57) ABSTRACT

A device EA that manufactures a projection-bearing body having a structure in which projections CV are formed on a bearing sheet BS comprises: a material transfer unit 10 that executes a material transfer step of transferring a base material BM containing a plastic material PM; a material support unit 20 that executes a material support step of supporting the base material BM transferred by the material transfer unit 10, by a support member 22 having a support surface 22A in which recesses 22B corresponding to the projections CV are formed; and a press unit 30 that executes a pressing step of pressing the base material BM in a direction toward the support surface 22A to fill the plastic material PM in the recesses 22B and forming the projections CV from the plastic material PM. In the base material BM, the plastic material PM is stacked on one surface of the bearing sheet BS. When transferring the base material BM, the material transfer unit 10 places the base material BM on the support member 22, with the plastic material PM in contact with the support surface 22A.

### BACKGROUND OF THE INVENTION

### Field of the Invention

The present invention relates to a projection-bearing body manufacturing method and a projection-bearing body manufacturing device, and for example, relates to a method and a device usable for microneedle manufacturing.

### Description of the Related Art

There is known a microneedle having a structure in which needle-like projections are formed on a sheet-like object. In a manufacturing process of the microneedle having such a structure, the microneedle is molded using an intaglio, and when a thermoplastic resin which is its material is placed on a support surface of the intaglio, two steps are required. One is a step of placing the thermoplastic resin on the support surface, and the other is a step of placing the sheet-like object on the thermoplastic material placed on the support surface.

### SUMMARY OF THE INVENTION

The present invention disclosed and claimed herein, in one aspect thereof, comprises a method of manufacturing a projection-bearing body having a structure in which a projection is formed on a bearing sheet. The method comprises: a base material preparing step of preparing a base material having a structure in which a plastic material is stacked on one surface of the bearing sheet; a material transfer step of transferring the base material prepared in the base material preparing step, the material transfer step placing the base material on a support member having a support surface in which a recess corresponding to the projection is formed, with the plastic material in contact with the support surface; a material support step of supporting the base material transferred in the material transfer step, by the support member; and a pressing step of pressing the base material in a direction toward the support surface to fill the plastic material in the recess and forming the projection from the plastic material.

Further scope of applicability of the present invention will become apparent from the detailed description given hereinafter. The detailed description and embodiments are only given as examples though showing preferred embodiments of the present invention, and therefore, from the contents of the following detailed description, changes and modifications of various kinds within the spirits and scope of the invention will become apparent to those skilled in the art.

### BRIEF DESCRIPTION OF THE DRAWINGS

The present invention will be fully understood from the following detailed description and the accompanying drawings. The accompanying drawings only show examples and are not intended to restrict the present invention. In the accompanying drawings:

FIG. 1A-FIG. 1D are explanatory views of a manufacturing device that executes a projection-bearing body manufacturing method according to one embodiment and explanatory views of the operation of the same device; and

FIG. 1E and FIG. 1F are explanatory views of modification examples.

### DETAILED DESCRIPTION

An embodiment will be hereinafter described based on the drawings.

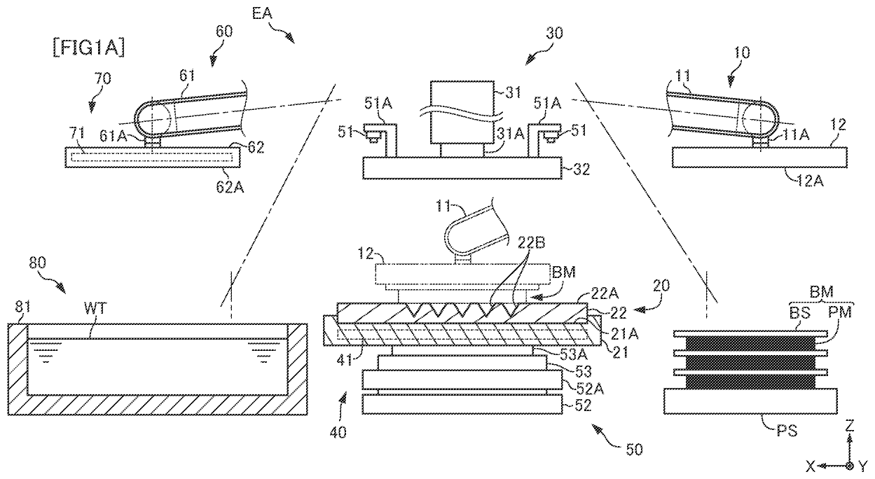

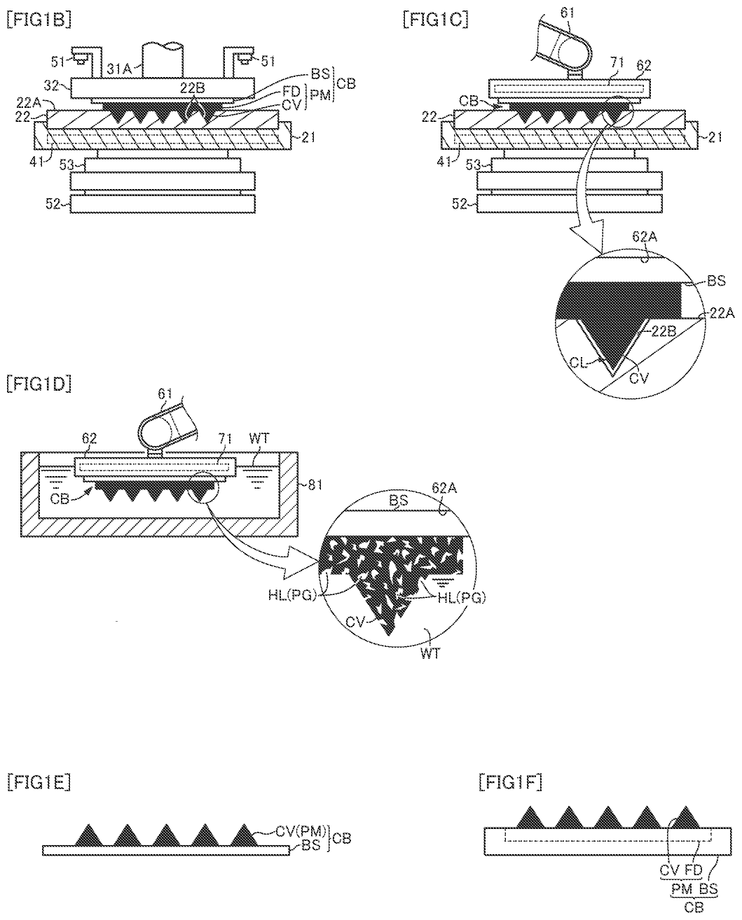

It should be noted that X-axis, Y-axis, and Z-axis in this embodiment are orthogonal to one another, where the X-axis and the Y-axis are within a predetermined plane while the Z-axis is orthogonal to the predetermined plane. Further, in this embodiment, FIG. 1A as viewed in the direction from the near side, which is parallel to the Y-axis, is used as a reference for direction, and when a direction is mentioned without any designation of a drawing, an “upper” direction means a direction indicated by an arrow along the Z-axis, a “lower” direction means a direction opposite the upper direction, a “left” direction means a direction indicated by an arrow along the X-axis, a “right” direction means a direction opposite the “left” direction, a “front” direction means a direction toward the near side in FIG. 1A in terms of a direction parallel to the Y-axis, and a “rear” direction means a direction opposite the “front” direction. Note that FIG. 1B to FIG. 1F where no arrow indicating a direction is given are all views seen from the same direction as FIG. 1A.

A projection-bearing body manufacturing device (hereinafter, also referred to simply as a “manufacturing device) EA is a device that executes a method of manufacturing a projection-bearing body CB in which projections CV are formed on a bearing sheet BS, and includes: a material transfer unit 10 that executes a material transfer step of transferring a base material BM containing a plastic material PM; a material support unit 20 that executes a material support step of supporting the base material BM transferred by the material transfer unit 10, by a support member 22 having a support surface 22A in which recesses 22B corresponding to the projections CV are formed; a press unit 30 that executes a pressing step of pressing the base material BM in a direction toward the support surface 22A to fill the plastic material PM in the recesses 22B and forming the projections CV from the plastic material PM; a heating unit 40 that executes a heating step of heating the plastic material PM; a positioning unit 50 that executes a positioning step of positioning the bearing sheet BS relative to the support surface 22A; a separating unit 60 that executes a separating step of separating the projection-bearing body CB from the support member 22; a cooling unit 70 that executes a cooling step of cooling the plastic material PM; and a leaching unit 80 that executes a leaching step of immersing the plastic material PM in water WT which is a leaching medium to turn the plastic material PM into a porous body. The manufacturing device EA is disposed near a base material supply unit PS that executes a base material supply step of supplying the base material BM.

Note that the base material BM has a structure in which the plastic material PM is stacked on one surface of the bearing sheet BS. Further, the bearing sheet BS of this embodiment is formed of paper which is a permeable member with permeability that allows at least liquid and gas to pass through. Further, the plastic material PM has thermoplasticity owing to polylactic acid (PLA) which is its main component, and contains polyethylene glycol PG (see the enlarged view in FIG. 1D) being a leaching material that leaches out in the water WT. The plastic material PM of this embodiment is formed of a mixture of two components, the polylactic acid and the polyethylene glycol PG, with their ratio being 5:5 in terms of weight ratio.

The material transfer unit 10 includes: what is called a multi-joint robot 11 as a drive device constituted by a plurality of arms and capable of displacing an object supported by its tip arm 11A which is a working part, to any position and any angle within its work range; and a holding plate 12 supported by the tip arm 11A and having a holding surface 12A capable of suction-holding owing to a not-illustrated pressure-reducing unit (holding unit) such as a pressure-reducing pump or a vacuum ejector. The material transfer unit 10 is configured to place the base material BM on the support member 22, with the plastic material PM in contact with the support surface 22A.

The material support unit 20 includes a base 21 in which a stepped part 21A is formed and the support member 22 fitted and supported in the stepped part 21A and having the recesses 22B formed in its support surface 22A.

The press unit 30 includes a direct-acting motor 31 as a drive device and a press member 32 supported by an output shaft 31A of the direct-acting motor 31. In this embodiment, the press member 32 is formed of a transparent material.

The heating unit 40 includes a heating device 41 constituted by, for example, a heating side of a coil heater or a heat pipe and disposed inside the base 21.

The positioning unit 50 includes detecting devices 51 constituted by imaging devices such as cameras or projectors or various sensors such as optical sensors or ultrasonic sensors and supported by the press member 32 through brackets 51A, an XY table 52 as a drive device, and a rotary motor 53 as a drive device supported by an output stand 52A of the XY table 52 and supporting the base 21 by its output shaft 53A. The positioning unit 50 is configured to move the support member 22 in an XY plane linearly or in a curved manner and also move the support member 22 in the XY plane in a rotating manner.

The separating unit 60 includes: what is called a multi-joint robot 61 as a drive device constituted by a plurality of arms and capable of displacing an object supported by its tip arm 61A which is a working part, to any position and any angle within its work range; and a holding plate 62 supported by the tip arm 61A and having a holding surface 62A capable of suction-holding owing to a not-illustrated pressure-reducing unit (holding unit) such as a pressure-reducing pump or a vacuum ejector.

The cooling unit 70 includes a cooling device 71 provided inside the holding plate 62 and constituted by, for example, a cooling side of a Peltier element or a heat pipe.

The leaching unit 80 includes the water WT and a vessel 81 that contains the water WT.

The operation of the projection-bearing body manufacturing device EA described above will be described.

First, a base material preparing step of preparing the base material BM in which the plastic material PM is stacked on one surface of the bearing sheet BS is executed. This base material preparing step is capable of preparing the base material BM by applying the plastic material PM in a liquid form on one surface of the bearing sheet BS using a known application device or by pasting the plastic material PM in a sheet form on one surface of the bearing sheet BS using a known sheet pasting device. When the preparation of the base material BM is completed, a user of the manufacturing device EA (hereinafter, simply referred to as a “user”) or a not-illustrated transfer unit such as a multi-joint robot or a belt conveyor supplies the base material BM onto the base material supply unit PS as illustrated in FIG. 1A, and thereafter the user inputs an automatic operation start signal to the manufacturing device EA in which its members are arranged at the initial positions indicated by the solid lines in FIG. 1A, through a not-illustrated operation unit such as an operation panel or a personal computer. In response, the material transfer unit 10 drives the multi-joint robot 11 and the not-illustrated pressure-reducing unit to bring the holding surface 12A into contact with the bearing sheet BS by moving the holding plate 12 and suction-hold the base material BM on the holding surface 12A, and places the base material BM on the support surface 22A of the support member 22 as indicated by the two-dot chain line in FIG. 1A. Thereafter, the material transfer unit 10 stops driving the not-illustrated pressure-reducing unit and returns the holding plate 12 to the initial position. Next, the press unit 30 drives the direct-acting motor 31 to move down the press member 32, and as illustrated in FIG. 1B, presses the base material BM in the direction toward the support surface 22A by the press member 32 to fill the plastic material PM in the recesses 22B. At this time, the heating unit 40 drives the heating device 41 to heat and soften the plastic material PM, thereby facilitating filling the plastic material PM in the recesses 22B. Thereafter, the positioning unit 50 drives the detecting devices 51 to detect the position of the bearing sheet BS, and based on this detection result, the positioning unit 50 drives the XY table 52 and the rotary motor 53 to move the support member 22 such that the recesses 22B (projections CV) are located at predetermined positions of the bearing sheet BS. Consequently, the projections CV and a foundation FD supporting the projections CV are formed from the plastic material PM, resulting in the formation of the projection-bearing body CB in which the projections CV are formed on the bearing sheet BS. Next, the press unit 30 drives the direct-acting motor 31 to return the press member 32 to the initial position, and thereafter, the heating unit 40 stops driving the heating device 41.

Then, the separating unit 60 and the cooling unit 70 drive the multi-joint robot 61 and the cooling device 71 respectively to bring the holding plate 62 into contact with the projection-bearing body CB and cool the plastic material PM as illustrated in FIG. 1C. Consequently, as illustrated in the enlarged view in FIG. 1C, the plastic material PM contracts, resulting in the formation of clearances CL between the plastic material PM and the recesses 22B, facilitating pulling off the projections CV from the recesses 22B. At this time, the cooling unit 70 cools the plastic material PM while the separating unit 60 presses the plastic material PM by the holding plate 62, enabling the good bonding of the plastic material PM and the bearing sheet BS. Next, when the cooling unit 70 stops driving the cooling device 71 to finish cooling the plastic material PM, the separating unit 60 drives the multi-joint robot 61 and the not-illustrated pressure-reducing unit to suction-hold the projection-bearing body CB on the holding surface 62A of the holding plate 62, thereby separating the projection-bearing body CB from the support member 22, and thereafter, as illustrated in FIG. 1D, immerses the projection-bearing body CB in the water WT. Consequently, the polyethylene glycol PG contained in the plastic material PM leaches out in the water WT, and as illustrated in the enlarged view in FIG. 1D, holes HL are formed in places where the polyethylene glycol PG has been present, and these places become void, so that the plastic material PM becomes porous. Thereafter, the separating unit 60 drives the multi-joint robot 61 to transfer the projection-bearing body CB having the holes HL to a not-illustrated subsequent step, stops driving the not-illustrated pressure-reducing unit to separate the holding plate 62 from the projection-bearing body CB, and returns the separated holding plate 62 to the initial position. Thereafter, the same operation as above is repeated.

According to the embodiment described above, since the base material BM in which the plastic material PM is stacked on one surface of the bearing sheet BS is placed on the support surface 22A, the material transfer step completes with one step. This can improve manufacturing capacity for manufacturing the projection-bearing body CB per unit time.

The invention is by no means limited to the above units and processes as long as the operations, functions, or processes described in these units and processes are achievable, still less to the above merely exemplary structures and processes described in the exemplary embodiment. For instance, the material support step may be any step as long as it is a step of supporting the base material transferred in the material transfer step, by the support member having the support surface in which the recesses corresponding to the projections are formed, and is by no means limited as long as it is within the technical scope in view of the common general technical knowledge at the time of the filing of the application (the same applies to the other units and steps).

The material transfer unit 10 may be configured to share the multi-joint robot 61 with the separating unit 60 to transfer the base material BM without employing the multi-joint robot 11, and may employ a suction pad or the like instead of or in addition to the holding plate 12.

The material support unit 20 may employ the base 21 not having the stepped part 21A, may have a pressure-reducing unit such as a pressure-reducing pump or a vacuum ejector or a support member holding unit such as a chuck motor, that holds the support member 22, and may be configured only with the support member 22 without the base 21.

The support member 22 may be formed of any material such as metal, resin, a silicone material, a rubber material, wood, paper, or pottery, and the number of the recesses 22B may be one or may be plural. A releasability improving member such as fluorine or silicone may be provided as a coating, applied, or stacked on the support surface 22A and the recesses 22B to facilitate separating the projection-bearing body CB from the support member 22.

The press unit 30 may press the base material BM in the direction toward the support surface 22A to form the projections CV by moving the support member 22 without moving the press member 32 or while moving the press member 32. The press member 32 may be translucent or may be non-transparent. The press unit 30 may share the multi-joint robot 11 and the holding plate 12 with the material transfer unit 10 or share the multi-joint robot 61 and the holding plate 62 with the separating unit 60, without employing the direct-acting motor 31 and the press member 32, to press the base material BM in the direction toward the support surface 22A through the holding plate 12 or the holding plate 62 to form the projections CV, or may share the multi-joint robot 11 with the material transfer unit 10 or share the multi-joint robot 61 with the separating unit 60 without employing the direct-acting motor 31, to press the base material BM in the direction toward the support surface 22A through the press member 32 to form the projections CV.

The heating unit 40 may be disposed outside the base 21 or may be disposed inside the support member 22 or inside the press member 32. The heating unit 40 may heat the plastic material PM before it is pressed by the press unit 30, may heat the plastic material PM while it is pressed by the press unit 30, or may heat the plastic material PM after it is pressed by the press unit 30. The projection-bearing manufacturing device EA of the present invention may be configured to include the heating unit 40 or may be configured not to include it.

The positioning unit 50 may move at least one of the support member 22 and the press member 32 in the XY plane linearly, in a curved manner, or in a rotating manner, and for example, may move the support member 22 in the X-direction (or the Y-direction) linearly or in a curved manner and move the press member 32 in the Y-direction (X-direction) linearly or in a curved manner, may move the support member 22 or the press member 32 in a direction other than the X-direction and the Y-direction linearly or in a curved manner, or may move the support member 22 or the press member 32 in the XY plane only linearly or in a curved manner or may move the support member 22 or the press member 32 in the XY plane only in a rotating manner.

The number of the detecting devices 51 may be one or may be two or more, and they may detect the position of the bearing sheet BS relative to the support surface 22A, from the sides of the press member 32. With the base 21 and the support member 22 being formed of a transparent or translucent material, the detecting devices 51 may detect the position of the bearing sheet BS relative to the support surface 22A by seeing it through the base 21 and the support member 22. The detecting devices 51 need not be supported by the press member 32.

The positioning unit 50 may perform the positioning of the plastic material PM relative to the support surface 22A or may perform the positioning of both the bearing sheet BS and the plastic material PM relative to the support surface 22A. The projection-bearing body manufacturing device EA of the present invention may be configured to include the positioning unit 50 or may be configured not to include it.

The separating unit 60 may share the multi-joint robot 11 with the material transfer unit 10 without employing the multi-joint robot 61, to separate the projection-bearing body CB from the support member 22, may paste a release tape on the projection-bearing body CB and apply tension to the release tape to separate the projection-bearing body CB from the support member 22, may employ a vibrating unit such as a vibrator or an ultrasonic vibrating device to apply vibration to the projection-bearing body CB, thereby facilitating separating the projection-bearing body CB from the support member 22, or may centrifuge the projection-bearing body CB. After immersing the projection-bearing body CB in the water WT, the separating unit 60 may move or vibrate the projection-bearing body CB to facilitate leaching out the polyethylene glycol PG in the water WT. The projection-bearing body manufacturing device EA of the present invention may be configured to include the separating unit 60 or may be configured not to include it. In the case where the separating unit 60 is not included therein, the projection-bearing body CB may be separated from the support member 22 by another device or manually.

The cooling unit 70 may have the cooling device 71 inside or near the press member 32 to cool the plastic material PM while the press member 32 presses the base material BM or cool the plastic material PM without the base material BM being pressed. The cooling unit 70 may be provided outside the holding plate 62 or may be provided inside the base 21 or the support member 22. The cooling unit 70 may cool the plastic material PM by immersing the plastic material PM in a cooling medium, for example, liquid such as water or a mixed solution, an organic solvent such as ethanol or acetone, or oil such as a mineral oil or a chemical synthetic oil, or may cool the plastic material PM by blowing atmospheric air, cold air, or the like to the plastic material PM. The cooling unit 70 may share the water WT with the leaching unit 80 without employing the cooling device 71, to cool the plastic material PM. The projection-bearing body manufacturing device EA of the present invention may be configured to include the heating unit 70 or may be configured not to include it.

The leaching unit 80 may employ any leaching medium as long as it allows the leaching material contained in the base material BM to leach out therein, and examples thereof include liquid such as water or a mixed solution, an organic solvent such as ethanol or acetone, or oil such as a mineral oil or a chemical synthetic oil. The leaching unit 80 may move, circulate, vibrate, or stir the leaching medium to facilitate leaching out the leaching material in the leaching medium. The projection-bearing body manufacturing device EA of the present invention may be configured to include the leaching unit 80 or may be configured not to include it.

The base material supply unit PS may be a simple table, a belt conveyor, a self-propelled transfer table, or the like. The projection-bearing body manufacturing device EA of the present invention may be configured to include the base material supply unit PS or may be configured not to include it.

The projection-bearing body manufacturing device EA may form the projections CV by pressing the base material BM in the direction toward the support surface 22A in a fluid atmosphere, for example, gas such as an elemental gas or a composite gas, liquid such as water or a mixed solution, an organic solvent such as ethanol or acetone, or oil such as a mineral oil or a chemical synthetic oil.

The bearing sheet BS may employ any permeable member as long as it allows at least one of liquid and gas to permeate through, for example, a fabric, a nonwoven fabric, resin, a mesh material, wood, or the like, and besides, metal, resin, a silicone material, a rubber material, wood, paper, pottery, or the like having through holes, or may employ a member not allowing liquid or gas to permeate through (an impermeable member), for example, metal, resin, a silicone material, a rubber material, wood, paper, pottery, or the like.

The plastic material PM may be any as long as it deforms when given energy such as heat, pressure, or the like and does not return to the original shape thereafter, that is, as long as it has plasticity. Examples thereof include metal, clay, and resin, and examples of the resin include PET, polypropylene, polyethylene, polyvinyl chloride, and a poly(lactide-co-glycolide) copolymer (PLGA). The plastic material PM may be composed of a single component or may be composed of a plurality of components. The plastic material PM may be composed of only a plastic component or may be composed of a plastic component and a non-plastic component. The plastic material may be one not having thermoplasticity and may be one not containing the leaching material. As the material having thermoplasticity, one containing a thermoplastic resin may be employed.

The ratio of the plastic material PM and the leaching material may be set to any ratio such as, for example, 8:2 or 1:9 instead of 5:5, and the ratio may be based on any such as a mass ratio, a volume ratio, or a molar ratio instead of the weight ratio.

The leaching material may be any as long as it leaches out in the leaching medium, and examples thereof include sodium chloride, potassium nitrate, alum, and polyvinyl alcohol.

The base material BM may be configured such that a substance other than the bearing sheet BS and the plastic material PM is stacked. The amount of the plastic material PM therein may be equal to the volume of the recesses 22B or may be more than the volume of the recesses 22B.

The height of the projections CV may be 100 nm to 3 mm, may be less than 100 nm, or may be more than 3 mm. In particular, in the case where they are used as what is called microneedles, the height of the projections CV is preferably 100 μm to 1.5 mm.

The tips of the projections CV may be pointed, non-pointed, round, arrowhead-shaped, or branched to two or three or more.

The shape of the projections CV may be a circular cone, a pyramid, a circular cylinder, a prism, or a combined shape of these.

The projection-bearing body CB may be one in which only the projections CV are formed from the plastic material PM and the projections CV are supported directly by the bearing sheet BS as illustrated in FIG. 1E, or may be one in which the projections CV and the foundation FD are formed from the plastic material PM and the foundation FD is buried in (penetrates in) the bearing sheet BS as illustrated in FIG. 1F.

The drive devices in the above-described embodiment each may be an electric machine such as a rotary motor, a direct-acting motor, a linear motor, a uniaxial robot, or a multi-joint robot having biaxial or triaxial or more joints, or may be an actuator such as an air cylinder, a hydraulic cylinder, a rodless cylinder, or a rotary cylinder. One in which some of these are directly or indirectly combined can also be employed.

In the above-described embodiment, in the case where one that presses an object to be pressed, such as a press unit or a press member such as a press roller or a press head, is employed, a member such as a roller, a round bar, a blade member, or a brush-shaped member may be employed or a structure that sprays a gaseous substance such as atmospheric air or gas may be employed, instead of or in addition to those exemplified in the above, and the one that presses may be formed of a deformable member such as rubber, resin, or sponge or may be formed of a non-deformable member such as metal or resin. In the case where one that supports (holds) a member to be supported (member to be held), such as a support (holding) unit or a support (holding) member, is employed, the member to be supported may be supported (held) by a gripping unit such as a mechanical chuck or a chuck cylinder, Coulomb force, an adhesive (adhesive sheet, adhesive tape), a tackiness agent (tacky sheet, tacky tape), magnetic force, Bernoulli suction, suction attraction, a drive device, or the like.

What is claimed is:

1. A method of manufacturing a projection-bearing body having a structure in which a projection is formed on a bearing sheet, the method comprising:
a base material preparing step of preparing a base material having a structure in which a plastic material is stacked on one surface of the bearing sheet;
a material transfer step of transferring the base material prepared in the base material preparing step, the material transfer step placing the base material on a support member having a support surface in which a recess corresponding to the projection is formed, with the plastic material in contact with the support surface;
a material support step of supporting the base material transferred in the material transfer step, by the support member; and
a pressing step of pressing the base material in a direction toward the support surface to fill the plastic material in the recess and forming the projection from the plastic material.

2. The method of claim 1,
wherein the plastic material has thermoplasticity, and
wherein the plastic material is heated on a preceding stage of the pressing step or in the pressing step.

3. The method of claim 1,
wherein the plastic material contains a leaching material that leaches out in a leaching medium, and
wherein the plastic material is turned into a porous body by being immersed in the leaching medium on a subsequent stage of the pressing step.

4. The method of claim 1,
wherein the plastic material is cooled on a subsequent stage of the pressing step.

5. The method of claim 1,
wherein the bearing sheet is positioned relative to the support surface in the pressing step.

6. The device of claim 2,
wherein the plastic material contains a leaching material that leaches out in a leaching medium, and
wherein the plastic material is turned into a porous body by being immersed in the leaching medium on a subsequent stage of the pressing step.

7. The method of claim 2,
wherein the plastic material is cooled on a subsequent stage of the pressing step.

8. The method of claim 2,
wherein the bearing sheet is positioned relative to the support surface in the pressing step.

9. The method of claim 3,
wherein the plastic material is cooled on a subsequent stage of the pressing step.

10. The method of claim 3,
wherein the bearing sheet is positioned relative to the support surface in the pressing step.

11. The method of claim 4,
wherein the bearing sheet is positioned relative to the support surface in the pressing step.

12. A device that manufactures a projection-bearing body having a structure in which a projection is formed on a bearing sheet, the device comprising:
a material transfer unit that transfers a base material having a structure in which a plastic material is stacked on one surface of the bearing sheet, the material transfer unit placing the base material on a support member having a support surface in which a recess corresponding to the projection is formed, with the plastic material in contact with the support surface;
a material support unit that supports the base material transferred by the material transfer unit, by the support member; and
a press unit that presses the base material in a direction toward the support surface to fill the plastic material in the recess and forms the projection from the plastic material.
